(19) United States
(12) Patent Application Publication
(10) Pub. No.: US 2019/0274937 A1
(43) Pub. Date: Sep. 12, 2019

(54) MICROBIOME-SAFE COSMETIC CLEANING COMPOSITIONS

(71) Applicants: Yun NV, Aartselaar (BE); Universiteit Antwerpen, Antwerpen (BE)

(72) Inventors: Tim Henkens, Aartselaar (BE); Filip Kiekens, Wilrijk (BE); Sarah Lebeer, Antwerpen (BE); Ingmar Claes, Antwerpen (BE)

(73) Assignees: Yun NV, Aartselaar (BE); Universiteit Antwerpen, Antwerpen (BE)

(21) Appl. No.: 16/347,267

(22) PCT Filed: Nov. 3, 2017
(86) PCT No.: PCT/EP2017/078131

§ 371 Date: May 3, 2019

(30) Foreign Application Priority Data
Nov. 4, 2016 (BE) 2016/5825

(51) Int. Cl. A61K 8/368; A61K 8/37; A61K 8/39; A61Q 19/10; A61Q 19/00

(52) U.S. Cl. CPC A61K 8/368; A61K 8/375; A61K 8/39; A61K 2800/596; A61Q 19/005; A61K 2800/524; A61K 2800/30; A61Q 19/10

(57) ABSTRACT

The present invention relates to microbiome-safe cosmetic cleaning compositions (i.e. soaps) and uses thereof in preserving the natural microflora of the human or animal skin/vagina. These cleaning compositions may also be beneficially used in combination with topical probiotic therapies, which may be compromised when using classical soaps. In particular, the present invention provides cosmetic cleaning compositions comprising at least one organic acid, as preservative; and at least one glycerol ester as surfactant. For these compositions it was found that they have the typical characteristics of a soap without the negative effects on the natural microflora of the skin/vagina.

### FIELD OF THE INVENTION

The present invention relates to microbiome-safe cosmetic cleaning compositions (i.e. soaps) and uses thereof in preserving the natural microflora of the human or animal skin/vagina. These cleaning compositions may also be beneficially used in combination with topical probiotic therapies, which may be compromised when using classical soaps. In particular, the present invention provides cosmetic cleaning compositions comprising at least one organic acid, as preservative; and at least one glycerol ester/ether as surfactant. For these compositions it was found that they have the typical characteristics of a soap without the negative effects on the natural microflora of the skin/vagina.

### BACKGROUND TO THE INVENTION

A soap mainly consists of an aqueous phase with surfactants, added for their cleansing and foaming purposes. A soap can additionally contain other excipients such as oils, antioxidants or preservatives for obtaining a longer shelf life of the product. Perfume is often added plentiful as well. Most ingredients of soaps can be harmful for bacteria and hereby the skin-microbiome as well. This could be problematic for the success rate of a topical treatment with probiotics. Hence the need for a ‘microbiome-safe’ soap. It was found that the combination of organic acids as preservative and glycerol ester/ether derivatives (such as ethoxylated glycerine derivates) as surfactants is a highly suitable combination in formulating a microbiome-safe soap.

### SUMMARY OF THE INVENTION

In a first aspect, the present invention provides a cosmetic cleaning composition comprising one or more organic acids and one or more polyoxyethylene glycerol fatty acid ester surfactants; wherein said composition has a pH of less than 7.0 and is substantially free of buffering agents; and preferably does not contain a sulphate-based surfactant.

In a particular embodiment, the compositions of the present invention have a pH of less than 5.5, preferably less than 5.0; more preferably less than 4.5.

In another particular embodiment, said one or more glycerol-ester surfactants are ethoxylated glycerine derivatives such as glycereth-2, glycereth-7, glycereth-26, glycereth 31, glycereth-2-cocoate, glycereth-7 cocoate, glycereth-17 cocoate, glycereth-26 cocoate, glycereth-17 tallowate, olive oil glycereth-8 esters, glycereth-7 triacetate, glycereth-25 PCA isostearate, glycereth-18 ethyl hexanoate, glycereth-7 benzoate, glycereth-7 diisononoate, glycereth-8 hydroxystearate, glycereth-6 laurate, glycereth-20 stearate, . . . more in particular selected from the list comprising glycereth-7-caprylate/caprate, glycereth-2-cocoate, glycereth-17-cocoate.

In yet a further embodiment, said one or more organic acids are selected from the list comprising benzoic acid, sorbic acid, citric acid, acetic acid, lactic acid, oxalic acid, formic acid, dehydroacetic acid, fumaric acid, gluconic acid, malic acid, succinic acid, tartaric acid, phosphoric acid and propionic acid or derivates hereof.

In a particular embodiment, the present invention provides a cosmetic cleaning composition of the invention; wherein said one or more glycerol-ester/ether surfactants are present at a concentration of about 1-about 90 wt %; more in particular about 10-about 40 wt %.

In yet a further embodiment, the present invention provides a cosmetic cleaning composition of the invention; wherein said one or more organic acids are present at a concentration of less than 2.5 wt %; more specific less than 1 wt %; even more specific less than 0.1 wt %.

In another embodiment, the cosmetic cleaning composition of the present invention may further comprise one or more components selected from the list comprising: gellants, antioxidants, humectants, hydrating components, emollients, active agents, and acidity regulators.

In yet a further embodiment, said one or more organic acids serve the purpose of preservative; and the cosmetic cleaning composition of the invention is substantially free of further preservatives.

In a very specific embodiment, the present invention provides a cosmetic cleaning composition; comprising at least 50 wt % water, 1-25 wt % glycerol ester/ether surfactant, 1-20 wt % co-surfactants, 0.01-0.5 wt % organic acids and 1-4.5 wt % emollients.

More specifically, the cosmetic cleaning composition of the present invention comprises at least 70 wt % water, 10-20 wt % glycerol ester/ether surfactant, 5-9.5 wt % co-surfactants, and 0.01-0.5 wt % organic acids.

In another particular embodiment, said one or more glycerol-ester/ether surfactants are ethoxylated glycerine derivatives such as glycereth-2, glycereth-7, glycereth-26, glycereth 31, glycereth-2-cocoate, glycereth-7 cocoate, glycereth-17 cocoate, glycereth-26 cocoate, glycereth-17 tallowate, olive oil glycereth-8 esters, glycereth-7 triacetate, glycereth-25 PCA isostearate, glycereth-18 ethylhexanoate, glycereth-7 benzoate, glycereth-7 diisononoate, glycereth-8 hydroxystearate, glycereth-6 laurate, glycereth-20 stearate, . . . more in particular selected from the list comprising glycereth-7-caprylate/caprate, glycereth-2-cocoate, glycereth-17-cocoate.

Alternatively, the present invention provides the use of the cosmetic cleaning compositions disclosed herein, in combination with probiotics and/or probiotherapy.

The present invention also provides cosmetic cleaning compositions as disclosed herein for use in preserving the natural microflora of the human or animal skin or vagina; or for use in combination with probiotics and/or probiotherapy.

In yet a further aspect, the present invention provides a method for preserving the natural microflora of the human or animal skin or vagina; said method comprising applying a cosmetic cleaning composition as defined herein to the human or animal skin or vagina.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Effect of commercial soaps on the growth of L. plantarum: The different soaps are compared against the reference growth curve (bacterial suspension not incubated or grown in the presence of soap—top red line). Soaps which have growth inhibiting properties will have growth curves with a later start of the log-phase (±8 hours for reference). Soaps with no growth-inhibiting or antibacterial properties will have growth curves similar to the reference.

FIG. 2: Effect of soaps of the invention containing organic acids and glycerol-ester surfactants on the growth of L. plantarum: The different soaps are compared against the reference growth curve (bacterial suspension not incubated or grown in the presence of soap). Soaps which have growth inhibiting properties will have growth curves with a later start of the log-phase (±8 hours for reference). Soaps with no growth-inhibiting or antibacterial properties will have growth curves similar to the reference.

### DETAILED DESCRIPTION OF THE INVENTION

In a first aspect, the present invention provides a cosmetic cleaning composition comprising one or more organic acids and one or more glycerol-ester/ether surfactants; wherein said composition has a pH of less than 7.0 and is substantially free of buffering agents; and preferably does not contain a sulphate-based surfactant.

In the context of the present invention, the term “cosmetic cleaning composition” is meant to be a soap or other kind of composition which is useful for cleaning (e.g. washing or sanitizing) human and/or animal mucosal surfaces such as the skin or the vagina. The cleaning compositions of the present invention are microbiome-safe, and therefore the term “cleaning composition” is not meant to include sterilizing and disinfecting compositions which have an anti-microbial function. Therefore, the compositions of the present inventions are preferably free from preservatives (other than the co-formulated organic acids) and other types of components which are typically used for the preservation of cosmetic compositions such as for example formaldehydes, halogen containing organic compounds, parabens, alcohols and antimicrobiol stabilizers (e.g. ehtylhexylglycerine, glycols, EDTA); more specifically phenoxyethanol, methyl parabens, propylparabens, isothiazolinones, methylisothiazolinones, benzylalcohol, . . . . More specifically, sodium laureth sulphate and derivatives hereof are a very common used surfactant and have a very strong bactericidal effect. Another commonly used surfactant, cocamidopropyl betaine and derivatives hereof also have a negative influence on the present lactobacillus. As strong and fast working preservatives; phenoxyethanol, parabens, bronopol, sulphites/sulphates, benzylalcohol and others were identified. Most allergens in perfume can also be identified as being harmful for bacteria. Therefore, the present invention, preferably does not contain such harmful components/preservatives as listed above.

In the context of the present invention, the term ‘organic acid’ is meant to be an organic compound with acidic properties. Evidently, in the context of the present invention, any suitable organic acid, can be used in as far as it can act as a preservative.

It was found that these organic acids have a slow working mechanism on microorganisms and that they do not immediately harm (probiotic) microorganisms once they come into contact with these organic acids. After longer exposure to the environment, such as the skin, the organic acids lose their activity due to a rising pH, thereby also not harming (probiotic) microorganisms over a longer period of time. While the invention is preferably performed using an organic acid as preservative (since these were found to lose their activity after exposure to the environment), it may also be performed by using another preservative in as far as it has a slow working mechanism, such as requires at least 24 h to become active.

With respect to the use of organic acids, the formulations are preferably substantially free from buffering agents. The presence of a buffering agent will keep the compositions at a low pH for a longer period of time, thereby taking longer to inhibit the preservative action of the organic acids, and thus increasing the risk of harming microorganisms once they come into contact with the organic acids. While most components will have a small buffering effect, it is desired to select the components of the compositions such that they do not substantially reduce or increase the time required for the inactivation of the organic acids.

Particularly suitable organic acids are those selected from the list comprising benzoic acid, sorbic acid, citric acid, acetic acid, lactic acid, oxalic acid, formic acid, dehydroacetic acid, fumaric acid, gluconic acid, malic acid, succinic acid, tartaric acid, phosphoric acid and propionic acid. Alternatively, each of these organic acids may be used in its corresponding salt form such as for example instead of benzoic acid, sodium benzoate may be used.

In a particular embodiment, the pH of the compositions according to the present invention is less than 5.5, preferably less than 5.0, more preferably less than 4.5, less than 4.0 or less than 3.5. The pH of the formulations is highly relevant within the context of the present invention. In general, the lower the pH, the higher its preservative effect, thereby contributing to the long-term preservation of the formulations of the present invention. The desired pH is obtained by the co-formulated organic acids, if needed further adjusted with other acids, which thus have the purpose of preservative agent in the formulations. Due to the presence of these organic acids, we found that there was no further need to include additional preservatives, hence, the formulation of the present invention is preferably substantially free of other preservatives than the organic acids.

In a particular embodiment, the present invention provides a cosmetic cleaning composition of the invention; wherein said one or more organic acids are present at a concentration of less than 5 wt % or 2.5 wt % more specific less than 1 wt %; even more specific less than 0.1 wt %.

In the context of the invention the term “glycerol-ester” is meant to be a compound having a glycerol backbone. Glycerides, or acylglycerols, are esters formed from glycerol and fatty acids. Glycerol has three hydroxyl groups which can be esterified with one, two, or three fatty acids to form monoglycerides, diglycerides, and triglycerides. The ester functional group yields a hydrophilic end and the fatty acid chain offers a lipophilic part. The glyceride part can vary in chain length, functional groups & hydrophilic parts, making for a variety of possible HLB values, defining the hydrophilic/lipophilic balance of the molecule and hereby the cleansing/emulsifying capacity of said molecule.

In a particular embodiment said glycerol-ester surfactants are ethoxylated glycerine derivatives; more in particular selected from the list comprising glycereth-7-caprylate/caprate, glycereth-2-cocoate, glycereth-17-cocoate.

In the context of the invention the term “surfactant” is meant to be a component that lowers the surface tension between two liquids or between a liquid and a solid. Surfactant may generally act as detergents, wetting agents, emulsifiers, foaming agents or dispersants. In the context of the present invention the surfactants preferably act as detergent & foaming agents.

In a particular embodiment, the present invention provides a cosmetic cleaning composition of the invention; wherein said one or more glycerol-ester surfactants are present at a concentration of about 1-about 90 wt %; more in particular about 10-about 40 wt %.

In another embodiment, the cosmetic cleaning composition of the present invention may further comprise one or more components selected from the list comprising: gellants, antioxidants, humectants, hydrating components, emollients, active agents, and acidity regulators. Evidently, any additional co-formulated component should be selected such that it thus not substantially harm the natural microbiome of the skin or vagina. Such additional components may be present at varying concentrations but if used, are present at the following concentrations: gellant (0.1-5 wt %), antioxidant (0.01-5 wt %), humectants (0.1-10 wt %), hydrating components/emollients (0.01-20 wt %), active substances (such as salicyl acid/lactic acid 0.1-2 wt %), acidity regulators (HCl/NaOH, lactic acid).

In another embodiment, the cosmetic cleaning composition of the present invention may further comprise one or more co-surfactants. They act together with the main surfactant (ethoxylated glycerine derivates) to enhance its properties. They can help in further lowering the surface tension and hereby help in a better cleansing capacity from the surface to cleanse. They can also help in making the main surfactant sufficiently soluble or help in forming a stable micro-emulsion. In the scope of the present invention it is important that the co-formulated co-surfactants are chosen so and that they are present in such concentrations that they do not show any significant antibacterial activity.

The present invention provides a cosmetic cleaning composition compromising at least 30% water, 1-40 wt % glycerol ester surfactant, 1-15 wt % co-surfactants, 0.01-1 wt % organic acids and 1-14 wt % emollients.

Specifically, the present invention provides a cosmetic cleaning composition; comprising at least 50 wt % water, 1-25 wt % glycerol ester surfactant, 1-20 wt % co-surfactants, 0.01-1 wt % organic acids and 1-4 wt % emollients.

More specifically, the present invention provides cosmetic cleaning composition; comprising at least 70 wt % water, 10-20 wt % glycerol ester surfactant, 5-9.5 wt % co-surfactants, and 0.01-0.5 wt % organic acids. Even more specifically, the present invention provides cosmetic cleaning composition; comprising at least 70 wt % water, 10-29.5 wt % glycerol ester surfactant, and 0.01-0.5 wt % organic acids.

In these specific embodiments, said one or more glycerol-ester/ether surfactants are ethoxylated glycerine derivatives such as glycereth-2, glycereth-7, glycereth-26, glycereth 31, glycereth-2-cocoate, glycereth-7 cocoate, glycereth-17 cocoate, glycereth-26 cocoate, glycereth-17 tallowate, olive oil glycereth-8 esters, glycereth-7 triacetate, glycereth-25 PCA isostearate, glycereth-18 ethyl hexanoate, glycereth-7 benzoate, glycereth-7 diisononoate, glycereth-8 hydroxystearate, glycereth-6 laurate, glycereth-20 stearate, . . . more in particular selected from the list comprising glycereth-7-caprylate/caprate, glycereth-2-cocoate, glycereth-17-cocoate.

Some preferred, but non-limiting examples of the compositions of the present invention include suspensions, emulsions, solutions, ointments, creams, lotions, which may be formulated with carriers, excipients, and diluents that are suitable per se for such formulations, such as lactose, dextrose, sucrose, sorbitol, mannitol, starches, gum acacia, alginates, tragacanth, gelatin, polyethylene glycol, cellulose, (sterile) water, methylcellulose, edible oils, vegetable oils and mineral oils or suitable mixtures thereof. The compositions of the present invention can be in liquid, solid or semi-solid form; preferably in liquid form. The present invention thus also provides a liquid cosmetic cleaning composition comprising one or more organic acids and one or more glycerol-ester surfactants; wherein said composition has a pH of less than 7.0 and is substantially free of buffering agents. The formulations of the present invention can optionally contain other components such as drugs and/or prebiotics, for example for stimulating the growth of microorganisms.

In a further aspect, the present invention provides the use of the cosmetic cleaning compositions disclosed herein for preserving the natural microflora of the human or animal skin or vagina. Alternatively, the present invention provides the use of the cosmetic cleaning compositions disclosed herein, in combination with probiotics and/or probiotherapy.

The present invention also provides cosmetic cleaning compositions as disclosed herein for use in preserving the natural microflora of the human or animal skin or vagina; or for use in combination with probiotics and/or probiotherapy.

In yet a further aspect, the present invention provides a method for preserving the natural microflora of the human or animal skin or vagina; said method comprising applying a cosmetic cleaning composition as defined herein to the human or animal skin or vagina.

### EXAMPLES

In these examples, the effect of several commercial soaps on the growth curves of L. plantarum (reference probiotic for possible topical applications) was compared with soaps of the invention containing glycerol ester derivatives and organic acids. While many commercial soaps include one or more organic acids, none of them includes glycerol ester derivatives as a main surfactant, and they mostly contain harmful preservatives such as phenoxyethanol, methylparabens, propylparabens, isothiazolinones, methylisothiazolinones& benzylalcohol and aggressive surfactants such as sodium laureth sulphate.

### Example 1: Survival of Probiotic Microorganisms in Commercial Soaps/Cleaning Compositions

### Materials and Methods

The tested commercial soaps were picked randomly from both supermarkets and pharmacies, which claim to have more ‘skin-friendly’ soaps. Both skin-soaps and vaginal washing soaps were tested. Vaginal washing soaps were selected because they often claim to balance the vaginal flora. This experiment evaluated whether these soaps could be used together with a potential topically applied lactobacillus species. If no growth retardation is measured, the soaps could be labeled as compatible with topically applied lactobacillus probiotics or ‘biome-safe’ products. The tested soaps are listed in Table 1.0.

TABLE 1.0
List of the tested commercial soaps.

| Well | Zeep |
|---|---|
| 1 | Dove - Men + care clean comfort |
| 2 | Axe- peace |
| 3 | Le petit Marseillais - extra doux vervine & citron |
| 4 | AH- men showergel 2 in 1 |
| 5 | A Derma - Surgrass - soap free |
| 6 | Louis widmer - Duschöl - sehr trockene haut |
| 7 | la roche posay - lipikar gel lavant - peuax sensible - pH |
| | physiologique - sans savon |
| 8 | Avene cleanance gel - soapless cleanser |
| 9 | Vichy - normaderm - deep cleansing purifying gel |
| 10 | Lactacyd antibacterial washing emulsion |
| 11 | Lactacyd sensitive - extra soft intimate washing lotion |
| 12 | SAUGELLE ACTIVE PH 3.5 - natural cleansing - irritation/ |
| | redness |
| 13 | Saugelle pH 4.5 - respects the intimate flora (from 4 year)) |
| 14 | Rituals - Sakura Spring - foaming shower oil |
| 15 | Magnolia badschuim - zonder zeep - pH neutral |
| 16 | Bodysol - dermo protect douchemousse - 100% zacht (orchidee |
| | sensation) |
| 17 | Garnier - ultra doux shampoo 2 in 1 (vanille - papaya) |
| 18 | Lactacyd pharma - intimate washing emulsion - helps in |
| | balancing the intimate flora |

A 10% (m/m) soap solution was made in physiological (0.85% NaCl) water. A bacterial suspension of L. plantarum was added to obtain a concentration of ±4*10\hat{ }7 CFU/ml. The solution was mixed and put to rest for 10 minutes to allow incubation of the present soap ingredients. As reference sample, a bacterial suspension of L. plantarum was added to physiological water to obtain a concentration of ±4*10\hat{ }7 CFU/ml, after which the solution was mixed and put to rest for 10 minutes. After resting for 10 min, for both the soap solution containing bacteria and the reference sample a 1/10 dilution was made in a honeycomb well-plate containing MRS pH 5.5. The wells contains a final 1% soap solution and a theoretical concentration of 4*10\hat{ }6 CFU/ml. The pH of 5.5 was chosen to be resembling a normal skin pH. The 1% remaining soap in the wells resembles a retention factor of 0.01 after showering as premised in the ‘Notes of guidance by the scientific committee on consumer safet (SCCS)’. The measurement of optical density—Bioscreen C® Type FP-1100C—was used to produce the growth curves. The well plate was shaken 10 seconds before measurements every 30 minutes at 37° C. at a wavelength of 600 nm.

### Results and Discussion

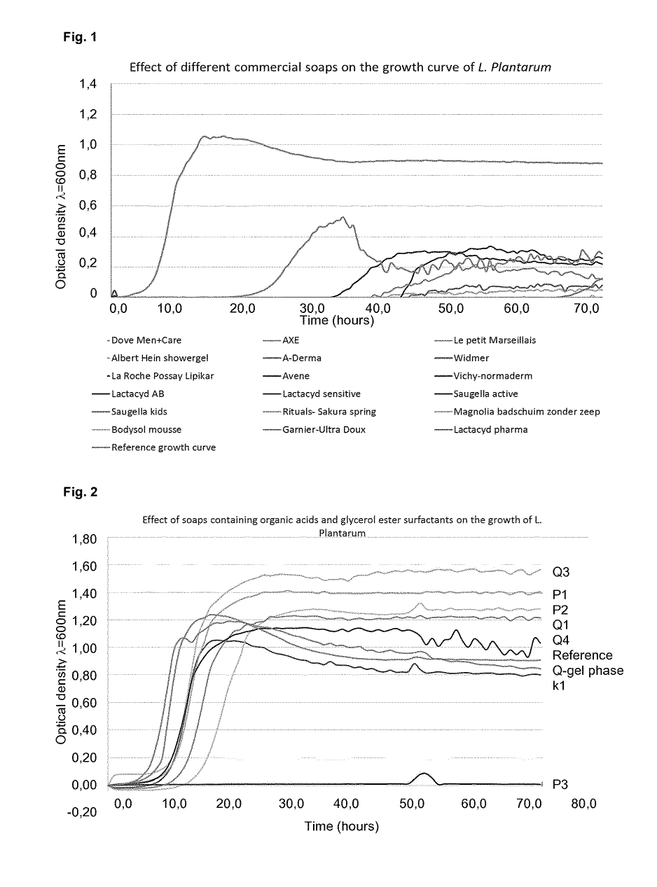

The max OD is a parameter which correlates with the growth density of the bacteria in the well. These are clearly limited for the commercial soaps (see FIG. 1). The log-phase (exponential growth phase of the tested bacteria) of all tested soaps starts at least three times after that of the reference (±8 vs±23 vs±40 hours), indicating a very huge growth retardation. Most of the tested soaps do not show any signs of growth at all. This result can of course be expected since cosmetics are required to pass a preservative challenge test, to prevent the growth of harmful organisms in the products and to prove that the preservative system is effective in controlling microbial contamination. Therefore, many formulators choose a mix of different fast and strong working preservatives to pass these challenge tests. This makes it of course difficult to introduce a topical therapy with probiotics, if they are killed afterwards by simple showering. It is clear that no normal growth curves were obtained using the tested soaps. Even the vaginal washes claiming a ‘restoring or balancing of the vaginal flora’, do not allow a normal growth for the added lactobacillus, whilst lactobacillus are a common species for the vaginal microbiome. This proves the need for a microbiome-safe soap.

Besides the preservatives having an inhibitory effect on the growth of micro-organisms, most included perfumes and surfactants have an inhibitory effect as well. This is most certainly true for the anionic, cationic and amphoteric molecules as those can easily bind with cell wall and cell membrane molecules of micro-organism.

A more detailed screening of the products (data not shown) led to a few common ingredients found in most shower cosmetics that can be held responsible for the growth retardation in FIG. 1.0. Sodium laureth sulphate and derivatives hereof are most common used and have a very strong bactericidal effect. Another commonly used surfactant, cocamidopropyl betaine and derivatives hereof also have a big negative influence on the present lactobacillus. As strong and fast working preservatives; phenoxyethanol, parabens, bronopol, sulphites/sulphates, benzylalcohol and others were identified. Most allergens in perfume can also be identified as being harmful for bacteria.

### Example 2: Survival of Probiotic Microorganisms in Soaps of the Invention

### Materials and Methods

Different soaps containing organic acids (Sodium Benzoate and/or Potassium sorbate) as preservative and glycerol-esters (glycereth-7caprylate/caprate; glycereth-2 cocoate; glycereth-7 cocoate) as main surfactants were formulated as detailed in Table 2.0.

TABLE 2.0
Formulas of different soaps according to the invention
containing the Organic Acid preservatives Sodium Benzoate
and/or potassium sorbate (and lactic acid if applicable)
and a glycerol-ester as main surfactant

| % | | |
|---|---|---|
| m/m | Type | INCI |
| | | K1 |
| 74.5 | | aqua |
| 1 | | glycerol |
| 1 | Preservative | sodium benzoate, potassium sorbate, aqua |
| 0.5 | | polyacrylate crosspolymer-6, t-butyl alcohol, |
| | | aqua |
| 10 | | coco-glucoside, glyceryl oleate, aqua |
| 5 | Glycerol ester | glycereth-7 caprylate/caprate |
| 5 | | PEG-200 hydrogenated glyceryl palmate, peg-7 |
| | | glyceryl cocoate, aqua |
| 3 | | cocamide mea |
| | | K5 |
| 48.5 | | aqua |
| 5 | | glycerol |
| 1 | Preservative | sodium benzoate |
| 0.5 | | polyacrylate crosspolymer-6, t-butyl alcohol, |
| | | aqua |
| 10 | | coco-glucoside, glyceryl oleate, aqua |
| 5 | | PEG-200 hydrogenated glyceryl palmate, peg-7 |
| | | glyceryl cocoate, aqua |
| 20 | | sucrose laurate, aqua, alcohol denat |
| 10 | Glycerol ester | glycereth-7 caprylate/caprate |
| | | P1 |
| 54.5 | | aqua |
| 1 | | glycerol |
| 1 | Preservative | sodium benzoate, potassium sorbate, aqua |
| 0.5 | | polyacrylate crosspolymer-6, t-butyl alcohol, |
| | | aqua |
| 10 | | coco glucoside, glyceryl oleate, aqua |
| 15 | Glycerol ester | glycereth-7 caprylate/Caprate |
| 5 | | PEG-200 hydrogenated glyceryl palmate, peg-7 |
| | | glyceryl cocoate, aqua |
| 3 | | cocamide mea |
| 10 | | sucrose laurate, aqua, alcohol denat |
| qs | Preservative/ | Lactic acid |
| | acidity | |
| | regulator | |
| | | P2 |
| 54.5 | | aqua |
| 1 | | glycerol |
| 1 | Preservative | sodium benzoate, potassium sorbate, aqua |
| 0.5 | | polyacrylate crosspolymer-6, t-butyl alcohol, |
| | | aqua |
| 10 | | coco glucoside, glyceryl oleate, aqua |
| 15 | Glycerol ester | glycereth-7 caprylate/Caprate |
| 5 | | PEG-200 hydrogenated glyceryl palmate, peg-7 |
| | | glyceryl cocoate, aqua |
| 3 | | cocamide mea |
| 10 | | Disodium cocoamphidoacetate, sodiumchloride, |
| | | aqua |
| qs | Preservative/ | Lactic acid |
| | acidity | |
| | regulator | |
| | | P3− Negative control |
| 65.9 | | aqua |
| 0.8 | | glycerol |
| 1 | Preservative | sodium benzoate, potassium sorbate, aqua |
| 0.4 | | polyacrylate crosspolymer-6, t-butyl alcohol, |
| | | aqua |
| 31.9 | | Sodium laureth sulfate, cocamidopropyl |
| | | betaine, coco glucoside, aqua |
| qs | Preservative/ | Lactic acid |
| | acidity | |
| | regulator | |
| | | Q1 |
| 57 | | aqua |
| 1 | | glycerol |
| 1 | Preservative | sodium benzoate, potassium sorbate, aqua |
| 1 | | polyacrylate crosspolymer-6, t-butyl alcohol, |
| | | aqua |
| 5 | | coco glucoside, glyceryl oleate, aqua |
| 20 | Glycerol ester | glycereth-7 caprylate/Caprate |
| 10 | | PEG-200 hydrogenated glyceryl palmate, peg-7 |
| | | glyceryl cocoate, aqua |
| 5 | Glycerol ester | Glycereth-2-cocoate |
| qs | Preservative/ | Lactic acid |
| | acidity | |
| | regulator | |
| | | Q3 |
| 57 | | aqua |
| 1 | | glycerol |
| 1 | Preservative | sodium benzoate, potassium sorbate, aqua |
| 1 | | polyacrylate crosspolymer-6, t-butyl alcohol, |
| | | aqua |
| 10 | | coco glucoside, glyceryl oleate, aqua |
| 15 | Glycerol ester | glycereth-7 caprylate/Caprate |
| 5 | | PEG-200 hydrogenated glyceryl palmate, peg-7 |
| | | glyceryl cocoate, aqua |
| 10 | | Cocamidopropyl hydroxysultaine, aqua |
| qs | Preservative/ | Lactic acid |
| | acidity | |
| | regulator | |
| | | Q4 |
| 57 | | aqua |
| 1 | | glycerol |
| 1 | Preservative | sodium benzoate, potassium sorbate, aqua |
| 1 | | polyacrylate crosspolymer-6, t-butyl alcohol, |
| | | aqua |
| 5 | | coco glucoside, glyceryl oleate, aqua |
| 12.5 | Glycerol ester | glycereth-7 caprylate/Caprate |
| 7.5 | | PEG-200 hydrogenated glyceryl palmate, peg-7 |
| | | glyceryl cocoate, aqua |
| 10 | Glycerol ester | Glycereth-2-cocoate |
| 5 | Glycerol ester | Glycereth 17-cocoate |
| qs | Preservative/ | Lactic acid |
| | acidity | |
| | regulator | |
| | | Q-GELPHASE |
| 57 | | aqua |
| 1 | | glycerol |
| 1 | Preservative | sodium benzoate, potassium sorbate, aqua |
| 1 | | polyacrylate crosspolymer-6, t-butyl alcohol, |
| | | aqua |

The pH of the soap was adjusted with either HCL/NaOH or lactic acid to 4.5±0.1. A pH of below 5.5 is needed for organic acids to be active as preservative. P3 was formulated as a negative control. This soap contains the most commonly used surfactants (Sodium laureth sulfate, cocamidopropyl betaine, coco glucoside) in commercially available soaps (See addendum 4.0). The Q-gel phase is to evaluate the effect of organic acids as preservatives in these formulations and to prevent interference of the gel-phase. See table 2.0 for formulas.

A 10% (m/m) soap solution was made in physiological (0.85% NaCl) water. A bacterial suspension of L. plantarum was added to obtain a concentration of ±4*10\hat{ }7 CFU/ml. The solution is mixed and put to rest for 10 minutes to allow incubation of the present soap ingredients. As reference sample, a bacterial suspension of L. plantarum was added to physiological water to obtain a concentration of ±4*10\hat{ }7 CFU/ml, after which the solution was mixed and put to rest for 10 minutes. After resting for 10 min, for both the soap solution and reference sample a 1/10 dilution is made in a well containing MRS pH 5.5. The wells contain 1% soap solution and a theoretical concentration of 4*10\hat{ }6 CFU/ml. The pH of 5.5 was chosen to be resembling a normal skin pH. The 1% remaining soap in the wells resembles a retention factor of 0.01 after showering as premised in the ‘Notes of guidance by the scientific committee on consumer safety (SCCS)’. The measurement of optical density—Bioscreen C® Type FP-1100C—will produce the growth curves. The well plate is shaken 10 seconds before measurements every 30 minutes at 37° C. at a wavelength of 600 nm.

### Results and Discussion

All the tested soaps of the invention show a normal growth curve (FIG. 2) in contrast to that of the commercial soaps (FIG. 1). The Log-phase initiated at ±8 hours for the reference curve where most soaps initiated the log-phase around ±8-10 hours. This is only a very slight delay and it can be said that this is a non-significant delay. Also the maximum obtained OD is alike for all the soaps and the reference curve.

The Q-gel-phase is a control to evaluate the effect of the organic acids and the gellant that are present in the q-series. It is clear that there is no effect as the curve matches the reference curve nearly perfectly. Therefore, any deviations from the reference curve can be attributed to the present surfactants and other excipients in the soap formulations. The organic acids are slow-working preservatives that need to penetrate the cell before being able to exert their function. After 10 minutes of incubation, the soap is diluted 1/10 in MRS pH5.5. This basically inactivates the organic acid, i.e. it will be >90% present in its non-active form (salt). It needs to be in its acid form to be lipophilic enough to penetrate the cell. This unique mechanism makes it a slow working, but effective preservative as long as the pH is maintained at 4.5. Application on the skin inactivates the organic acid and the local micro-organisms will influence no negative effect of the present organic acids in their salt form.

The negative control (P3) has no growth after incubation in MRS. It contains the anionic Sodium Laureth sulphate (14% final) and cocamidopropyl betaine, some of the most commonly used surfactants in present-day soaps. Their growth inhibiting effect can be attributed to their anionic bactericidal effect as tested through spread plating. This again indicates that most commercial soaps can have a negative influence on the present bacteria on the skin by either killing them or severely inhibiting their growth.

It can be concluded that the combination of organic acid preservatives and glycerol ester surfactants is a viable strategy in formulating soaps that are safe for the present micro-organisms (lactobacillus in this case) and can therefore be labeled as ‘biome-safe’.

1. A cosmetic cleaning composition comprising one or more organic acids and one or more glycerol-ester surfactants; wherein the composition has a pH of less than 7.0; is substantially free of buffering agents and does not contain a sulphate-based surfactant.

2. A cosmetic cleaning composition according to claim 1; wherein the composition has a pH of less than 5.5.

3. A cosmetic cleaning composition according to claim 1; wherein the one or more glycerol-ester surfactants are ethoxylated glycerine derivatives.

4. A cosmetic cleaning composition according to claim 3; wherein the ethoxylated glycerine derivatives are selected from the group consisting of glycereth-2, glycereth-7, glycereth-26, glycereth 31, glycereth-2-cocoate, glycereth-7 cocoate, glycereth-17 cocoate, glycereth-26 cocoate, glycereth-17 tallowate, olive oil glycereth-8 esters, glycereth-7 triacetate, glycereth-25 PCA isostearate, glycereth-18 ethylhexanoate, glycereth-7 benzoate, glycereth-7 diisononoate, glycereth-8 hydroxystearate, glycereth-6 laurate, and glycereth-20 stearate.

5. A cosmetic cleaning composition according to claim 1; wherein the one or more organic acids are selected from the group consisting of benzoic acid, sorbic acid, citric acid, acetic acid, lactic acid, oxalic acid, formic acid, dehydroacetic acid, fumaric acid, gluconic acid, malic acid, succinic acid, tartaric acid, phosphoric acid, propionic acid and derivatives thereof.

6. A cosmetic cleaning composition according to claim 1; wherein the one or more glycerol-ester surfactants are present at a concentration of about 1-about 90 wt %.

7. A cosmetic cleaning composition according to claim 1; wherein the one or more organic acids, acting as preservative, are present at a concentration of less than 2.5 wt %.

8. A cosmetic cleaning composition according to claim 1; further comprising one or more components selected from the group consisting of gellants, antioxidants, ion scavengers, humectants, hydrating components, emollients, active agents, and acidity regulators.

9. A cosmetic cleaning composition according to claim 1; wherein the one or more organic acids serve the purpose of preservative; and wherein the composition is substantially free of further preservatives.

10. A cosmetic cleaning composition according to claim 1; comprising at least 30 wt % water, 1-40 wt % glycerol ester surfactant, 1-15 wt % co-surfactants, 0.01-1 wt % organic acids and 1-14 wt % emollients.

11. A cosmetic cleaning composition according to claim 1; comprising at least 50 wt % water, 10-40% glycerol ester surfactant, 1-9 wt % co-surfactants, and 0.01-1 wt % organic acids.

12. A cosmetic cleaning composition according to claim 1; comprising at least 70 wt % water, 10-20 wt % glycerol ester surfactant, 5-9.5 wt % co-surfactants, and 0.01-0.5 wt % organic acids.

13. A cosmetic cleaning composition according to claim 10, wherein the glycerol ester surfactant is a glycereth ester surfactant.

14.-18. (canceled)

19. A method for preserving the natural microflora of a human or animal skin or vagina, the method comprising applying a cosmetic cleaning composition according to claim 1 to the human or animal skin or vagina.

20. The method of claim 19, further comprising applying probiotics and/or probiotherapy in combination with the cosmetic cleaning composition to the human or animal skin or vagina.

21. A cosmetic cleaning composition according to claim 1, wherein the composition has a pH of less than 5.0

22. A cosmetic cleaning composition according to claim 3, wherein the ethoxylated glycerine derivatives are selected from the group consisting of glycereth-7-caprylate/caprate, glycereth-2-cocoate, and glycereth-17-cocoate.

23. A cosmetic cleaning composition according to claim 1, wherein the one or more glycerol-ester surfactants are present at a concentration of about 10-about 40 wt %.

24. A cosmetic cleaning composition according to claim 1, wherein the one or more organic acids, acting as preservative, are present at a concentration of less than 1 wt %.

25. A cosmetic cleaning composition according to claim 10, wherein the glycerol ester surfactant is a glycereth ester surfactant selected from the group consisting of glycereth-7-caprylate/caprate, glycereth-2-cocoate, and glycereth-17-cocoate.
